(19) United States
(12) Patent Application Publication
(10) Pub. No.: US 2026/0157885 A1
(43) Pub. Date: Jun. 11, 2026

(54) ARTIFICIAL INTELLIGENCE PLATFORM FOR SURGICAL VIDEO CLASSIFICATION

(71) Applicant: Alcon Inc., Fribourg (CH)

(72) Inventors: Zhuoran WU, Ranch Cucamonga, CA (US); Lu YIN, Keller, TX (US); Vignesh SURESH, Woodland Hills, CA (US); Ramesh SARANGAPANI, Coppell, TX (US); Joseph WEATHERBEE, San Clemente, CA (US); Kevin Michael BULGARELLI, Burleson, TX (US)

(21) Appl. No.: 19/414,856

(22) Filed: Dec. 10, 2025

Related U.S. Application Data
(60) Provisional application No. 63/730,854, filed on Dec. 11, 2024.

(51) Int. Cl. A61F 9/007; A61B 3/00; A61B 3/13; A61B 3/14; G06V 10/14; G06V 10/764; G06V 20/40; G16H 20/40

(52) U.S. Cl. CPC A61F 9/00745; A61B 3/0025; A61B 3/13; A61B 3/145; G06V 10/14; G06V 10/764; G06V 20/41; G06V 20/46; G06V 20/49; G16H 20/40; G06V 2201/03

(57) ABSTRACT

A system includes an ophthalmic microscope configured to capture video of an ophthalmic treatment. A computer system receives the video and a treatment plan for the ophthalmic treatment and processes the video and the treatment plan using a machine learning model to divide the video into a plurality of video segments, each video segment of the plurality of video segments corresponding to a procedure of a plurality of procedures included in the ophthalmic treatment. The computer system may process the video using a machine learning model to label each frame of a plurality of frames of the video with an identifier of a procedure of a plurality of procedures included in the ophthalmic treatment represented in each frame and at least one of (a) select information for display on the display device according to the identifier and (b) control operation of surgical equipment according to the identifier.

### CROSS-REFERENCE TO RELATED APPLICATIONS

This application claims the benefit of U.S. Provisional Application Ser. No. 63/730,854 (filed on Dec. 11, 2024), the content of which is incorporated by reference herein in its entirety.

### BACKGROUND

The present disclosure relates generally to providing imaging during ophthalmic surgery, such as cataract surgery, glaucoma surgery, or the like.

The human eye receives light through a clear outer portion called the cornea and focuses the resulting image by way of an ocular crystalline lens onto the retina. The quality of the focused image depends on many factors including the size and shape of the eye, and the transparency of the cornea and lens. When age or disease causes the lens to become less transparent, vision deteriorates because of the diminished light that is transmitted to the retina. This deficiency in the lens of the eye is medically known as a cataract. In addition, the crystalline lens may lose accommodation skills with age, which is called presbyopia. An accepted treatment for those conditions is the surgical removal of the crystalline lens followed by a replacement by an artificial intraocular lens (IOL).

Glaucoma is a group of eye diseases affecting the retina and optic nerve. Glaucoma is one of the leading causes of blindness worldwide. Most forms of glaucoma result when the intraocular pressure (IOP) increases to pressures above normal for prolonged periods of time. IOP can increase due to high resistance to the drainage of the aqueous humor relative to its production. Left untreated, an elevated IOP causes irreversible damage to the optic nerve and retinal fibers resulting in a progressive, permanent loss of vision.

Glaucoma is often treated by inserting an instrument through the cornea in order to make an incision or place a shunt or incision in the anterior chamber to facilitate drainage of fluid from the anterior chamber. A shunt may be placed, for example, in the trabecular meshwork, Schlemm's canal, suprachoroidal space, or elsewhere. During the treatment, the surgeon will view the anterior chamber and the instrument through gonioscope or an ophthalmic microscope in order to place the incision or shunt at an appropriate location with the application of an appropriate amount of pressure.

It would be an advancement in the art to facilitate the performance of cataract surgery, glaucoma surgery, and other ophthalmic treatments.

### SUMMARY

In certain embodiments, a system includes an ophthalmic microscope configured to capture video of an ophthalmic treatment. A computer system is coupled to the ophthalmic microscope and is configured to: receive a treatment plan for the ophthalmic treatment; and process the video and the treatment plan using a machine learning model to divide the video into a plurality of video segments, each video segment of the plurality of video segments corresponding to a procedure of a plurality of procedures included in the ophthalmic treatment.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1 illustrates an example operating environment for providing ophthalmic treatments in accordance with certain embodiments.

FIG. 2 illustrates frames of video captured during an ophthalmic treatment that may be processed in accordance with certain embodiments.

FIG. 3 is a schematic diagram of a machine learning system for labeling segments of video captured during an ophthalmic treatment in accordance with certain embodiments.

FIG. 4 is a schematic diagram of a temporal video segmentation model in accordance with certain embodiments.

FIG. 5 is a schematic diagram of a machine learning model for labeling streaming video in accordance with certain embodiments.

FIG. 6 is a process flow diagram of a method for using labeled video of an ophthalmic treatment in accordance with certain embodiments.

FIG. 7 is a process flow diagram of a method for using labeled video to monitor a phacoemulsification procedure in accordance with certain embodiments.

FIG. 8 is a plot of pupil size over time during a phacoemulsification procedure in accordance with certain embodiments.

FIG. 9 is a process flow diagram of a method for providing guidance during cataract surgery in accordance with certain embodiments.

FIGS. 10A to 10C are example overlays that may be displayed during cataract surgery in accordance with certain embodiments.

FIG. 11 is a process flow diagram of a method for providing post-operative visualization using labeled video in accordance with certain embodiments.

FIG. 12 is a schematic representation of a dashboard for presenting post-operative visualization using labeled video in accordance with certain embodiments.

FIG. 13 is a schematic diagram illustrating a system for labeling surgical video and using labeled surgical video in accordance with certain embodiments.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

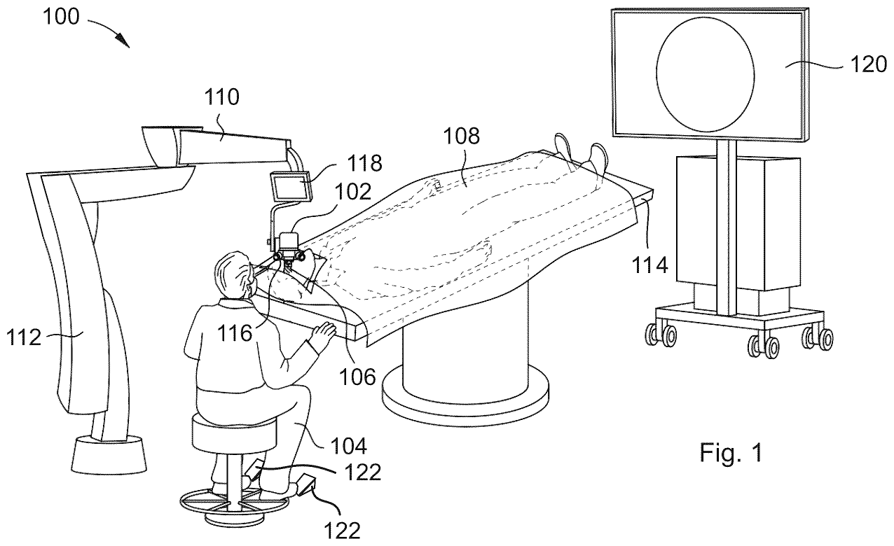

FIG. 1 illustrates an example system 100 that may be used to capture video that is labeled according to the approach described herein. The system 100 includes an ophthalmic microscope 102. A surgeon 104 uses the ophthalmic microscope 102 to visualize structures on and in an eye 106 of a medical patient 108 undergoing a surgery. The ophthalmic microscope 102 is supported on, in this illustration, an adjustable overhead arm 110 of a microscope support pedestal 112. The patient 108 may be supported on an operating table 114. The ophthalmic microscope 102 is movable with the overhead arm 110 in three dimensions so that the surgeon 104 can position the ophthalmic microscope 102 as desired with respect to the eye 106 of the patient 108.

In certain embodiments, the ophthalmic microscope 102 comprises a high resolution, high contrast stereo viewing ophthalmic microscope. The ophthalmic microscope 102 will often include a binocular (or monocular) eyepieces 116, through which the surgeon 104 will have an optically magnified view of the relevant eye structures that the surgeon 104 will need to see to accomplish a given surgery or diagnose an eye condition of the patient 108.

The ophthalmic microscope 102 includes a digital camera and broadband light source for capturing color (red, green, and blue) images, a multi-spectral imaging (MSI) device, and/or other type of imaging device. Digital images captured using the camera may be displayed on a display device within the ophthalmic microscope 102.

The ophthalmic microscope 102 may include two display devices viewable through binocular eyepieces 116 and that display images of the patient's eye 106 that are captured from different viewpoints by two cameras to provide stereoscopic viewing. For example, the ophthalmic microscope 102 may be implemented as the NGENUITY 3D VISUALIZATION SYSTEM provided by Alcon Inc. of Fort Worth Texas.

Images from the ophthalmic microscope 102 may be additionally or alternatively displayed on one or more display devices. For example, the one or more display devices may include a display device 118 fastened to the overhead arm 110 above the ophthalmic microscope 102.

In order to relieve the surgeon 104 from the need to constantly look into the binocular eyepieces 116 to obtain a stereoscopic view, the one or more display devices may include a display device 120 may be implemented as a three-dimensional display device. The display device 120 may therefore provide a stereoscopic view of images captured using the ophthalmic microscope 102. The display device 120 may be embodied as any type of three-dimensional display device known in the art, including those that do or do not use special filtering glasses. For some types of three-dimensional display devices, the perception of three dimensions requires that the distance of the viewer from the display device 120 be within a threshold distance from the display device. The display device 120 may be mounted to a cart, a manually adjustable or robotic arm, or other manually or automatically adjustable support.

Operation of the ophthalmic microscope 102, surgical instruments (e.g., phaco-vit instruments used in conjunction with a phaco-vit surgical console), and/or information displayed on the display devices 118, 120 may be controlled using foot pedals 122 operatively coupled to the ophthalmic microscope 102 and/or display devices 118, 120.

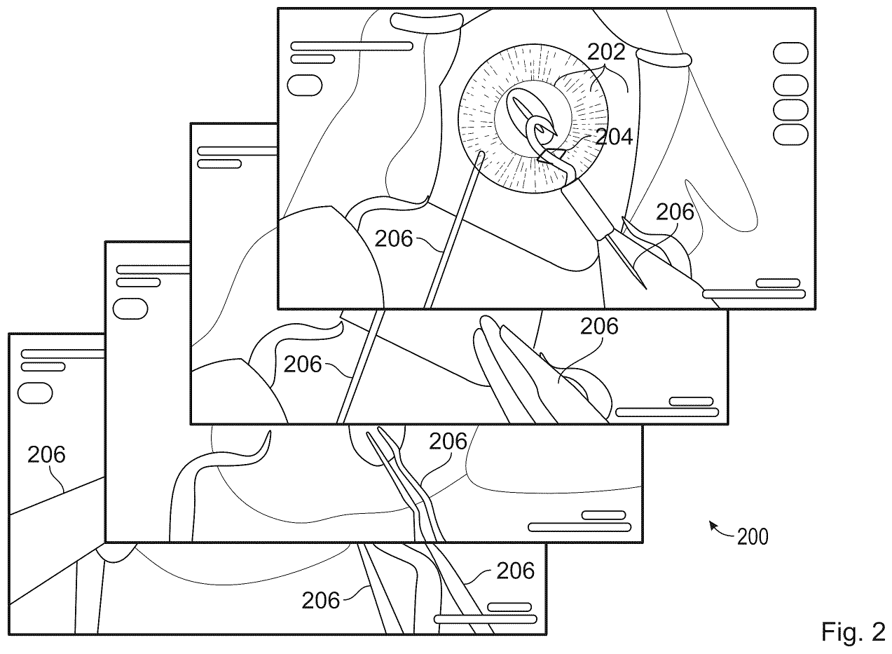

FIG. 2 illustrates images 200 that may be captured using a camera incorporated into the ophthalmic microscope 102. The images 200 may be frames of video captured using the camera. Accordingly, the images 200 may be arranged in sequence in order of capture. As used herein, an image 200 may be understood as being any of (a) a single (e.g., monocular) image, (b) a pairs of images captured using binocular cameras, such as those provided by the NGENUITY 3D VISUALIZATION SYSTEM, e.g., each pair of images captured at substantially (e.g., within 50 milliseconds) the same time, or (c) a volumetric (e.g., three-dimensional image) obtained from binocular images or other three-dimensional imaging modality.

The illustrated images 200 show the anatomy 202 of the eye, such as the iris, cornea, retina, and sclera. The anatomy 202 may further show the effect of actions performed by a surgeon 104, e.g., incisions 204, rhexis, phacoemulsification, lens implantation, or the like. The images 200 may further show portions of instruments 206 used during an ophthalmic treatment, such as scalpels, phaco-vit tools, lens insertion tools, aspirators, light sources, a gonioscope, or the like.

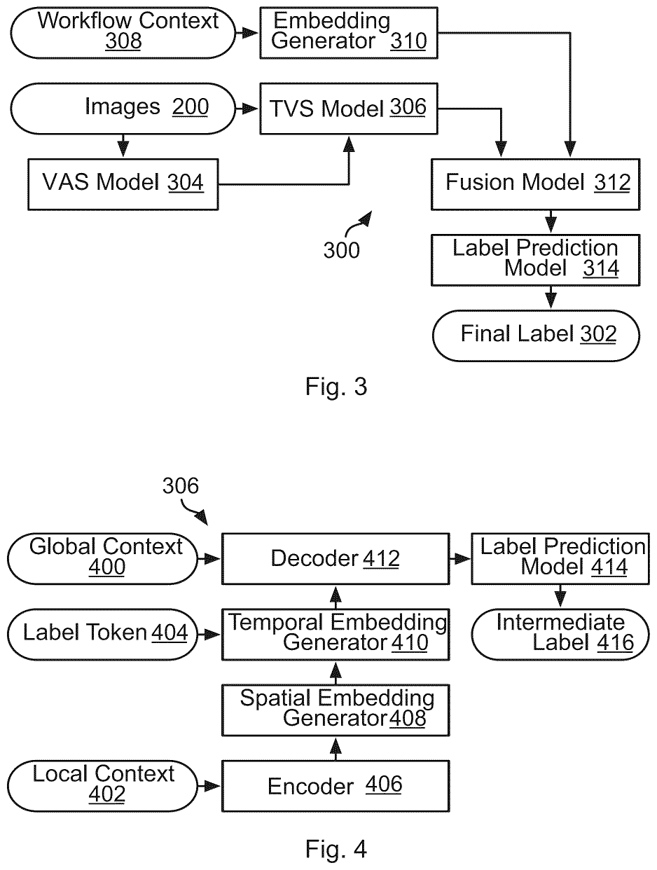

Referring to FIG. 3, images 200 may be processed using the illustrated machine learning model 300 that assigns final labels 302 to the images 200. The final labels 302 may indicate (a) a procedure of a treatment during which an image 200 was captured and (b) the procedure to which a group of consecutive images, i.e., a video segment, correspond.

The machine learning model 300 may include a video action segmentation (VAS) model 304 that processes each image of the images 200. The VAS model 304 is trained to identify what action is being performed in a particular image 200 within a video by processing an individual image 200 or a set of consecutive images 200 including the particular image 200. The VAS model 304 may be trained with a finite set of labels corresponding to a plurality of procedures included in one or more treatments. For example, a separate VAS model 304 may be trained and used for each type of treatment or a single VAS model 304 may be trained with labels for each procedure of a plurality of treatments. The VAS model 304 may be any machine learning model known in the art and may use any approach for performing VAS known in the art, such as a neural network, deep neural network (e.g., MAMBA network), convolution neural network (including a three-dimensional convolution neural network), recurrent neural network, transformer, multiple linear regression model, random sample consensus regression model, multiple polynomial regression model, support vector regression model, Bayesian neural network, genetic algorithm, long short term memory (LSTM) model, or other type of machine learning model.

Training data for the VAS model 304 may include video files having each frame labeled with the procedure that was being performed when each frame was captured. Where the VAS model 304 is trained to label procedures for multiple treatments, each frame may further be labeled with the treatment that was being provided when each frame was captured. The VAS model 304 may be trained with the training data to output a procedure label for a given image as an input either alone or as a set of multiple consecutive images from a video file.

The images 200 and labels for the images 200 as obtained using the VAS model 304 may be input into a temporal video segmentation (TVS) model 306. The TVS model 306 likewise outputs an intermediate label for each image 200 but takes into account additional information that overcomes some of the limitations of the VAS model 304. For example, the TVS model 306 may be implemented as the TVS model 306 illustrated in FIG. 4.

The accuracy of labels applied to each image 200 may further be enhanced using information in a workflow context 308. The workflow context 308 may, for example, include notes prepared by a surgeon in advance of performing a surgery. The notes may describe intended procedures to be performed during an ophthalmic treatment, a description of the condition of the eye 106, or other information. The workflow context 308 may include a treatment plan. For example, the treatment plan may be a data object processed by the ophthalmic microscope 102 to provide guidance during the ophthalmic treatment and/or configure parameters of the ophthalmic microscope 102 and/or other surgical equipment during each procedure of the ophthalmic treatment. The treatment plan may define other parameters of the ophthalmic treatment such as whether TRYPAN blue is used, whether the ophthalmic treatment is a regular or dense cataract surgery, description of complicating factors, parameters describing a minimally invasive glaucoma surgery (MIGS), or other parameters. The workflow context 308 may include transcriptions (speech-to-text) of audio statements made by the surgeon during the ophthalmic treatment.

The workflow context 308 may be processed by an embedding generator 310. The embedding generator 310 processes the data in the workflow context 308 to obtain a vector or array of embeddings. Embeddings are coded representations of the workflow context 308 that may be used by another stage of the machine learning model 300. The embedding generator 310 may be a neural network, deep neural network (e.g., MAMBA network), convolution neural network (including a three-dimensional convolution neural network), recurrent neural network, transformer, multiple linear regression model, random sample consensus regression model, multiple polynomial regression model, support vector regression model, Bayesian neural network, genetic algorithm, long short term memory (LSTM) model, or other type of machine learning model.

The embedding generator 310 may be an encoder. For example, the embedding generator 310 may be trained in as the encoder of an encoder-decoder system in which an encoder receives an input and generates an encoding and the decoder receives the encoding and attempts to recreate the input. The encoder is trained to encode sufficient information in the encoding to enable the decoder to recreate the input and the decoder is trained to use the encoding to recreate the input. The output of such an encoder may therefore be used embedding generator 310 and the encoding for a given input that is output by the encoder being used as the embedding. In yet another alternative, the embedding as output by the embedding generator may be the output of an internal (e.g., hidden) layer of a machine learning model trained to perform a task with respect to the workflow context, such as a classification task.

The embedding output by the embedding generator 310 and the intermediate label as output by the TVS model 306 may be processed by a fusion model 312. The fusion model 312 combines the label and the embedding into an intermediate representation, e.g., a vector or array of values. The intermediate representation may be input to a label prediction model 314, which outputs the final label 302. The final label 302 may be a word, code, or other value that identifies a procedure. The final label 302 may be a path through a hierarchy. For example, the hierarchy may have any number of levels such as treatment, procedure, sub-procedure, and possibly one or more additional levels. A sub-procedure may represent a movement, action, step, treated region, or other constituent part of a procedure. The final label 302 may therefore be in the form of [treatment name][procedure name][sub-procedure name]. For example, for cataract surgery, the final label 302 may be [cataract]->[rhexis]->[tear] or [cataract]->[rhexis]->[pull flap]->[medial quadrant], etc.

Labels as output by the VAS model 304 and TVS model 306 may be in the same form or a different form. Where the machine learning model 300 is trained for a specific ophthalmic treatment, the final label 302, and possibly other labels as output by the VAS model 304 and TVS model 306, may omit any identifier of an ophthalmic treatment.

The fusion model 312 and label prediction model 314 may be any machine learning model known in the art such as a neural network, deep neural network (e.g., MAMBA network), convolution neural network (including a three-dimensional convolution neural network), recurrent neural network, transformer, multiple linear regression model, random sample consensus regression model, multiple polynomial regression model, support vector regression model, Bayesian neural network, genetic algorithm, long short term memory (LSTM) model, or other type of machine learning model. The fusion model 312 and/or label prediction model 314 may be embodied as encoders according to any approach known in the art.

The embedding generator 310, TVS model 306, fusion model 312, and label prediction model 314 may be trained together to implement the functionality of the machine learning model 300. The VAS model 304 may be trained separately or may likewise be trained with the embedding generator 310, TVS model 306, fusion model 312, and label prediction model 314.

Training data entries may each include a workflow context 308, an image 200, and a human-generated final label. Inasmuch as there may be many minutes of video for a treatment, a single treatment may yield many thousands of training data entries, which may have the same workflow context. A training data entry may be processed using the machine learning model 300 to obtain a final label 302. The final label 302 output by the machine learning model 300 may be compared to the final label 302 of the training data entry and parameters of the machine learning model 300 may be updated according to the comparison.

FIG. 4 illustrates an example implementation of the TVS model 306. Inputs to the TVS model 306 for an image 200 (“the subject image 200”) may include a global context 400, local context 402, and a label token 404. The label token 404 may be the output of the VAS model 304 resulting from processing the subject image 200.

The global context 400 of the subject image 200 may include a first set of consecutive images including the subject image 200, such as the last image 200 of the first set of consecutive images or at some intermediate position within the first set of consecutive images.

In some embodiments, the first set of images of the global context 400 is a set of non-consecutive images selected from the frames of a video file. The set of images may represent an entire surgical flow from a start of a surgery until the last image 200 in the video file. The number of images in the global context 400 may be fixed or increase with time as the number of frames in the video file increases. Frames from the video file may be added to the global context based on a fixed interval (every N^{th} frame preceding the last frame of the video file) or based on prior labeling: the first frame (or other sequence number) of each previously labeled segment of the video filed that is labeled according to any of the approaches described herein.

The global context 400 may be used directly or may be processed using an encoder with the output of the encoder being used as the global context 400 as described below. For example, the global context 400 as used below may be replaced with the output of an encoder embodied as a long short term memory (LSTM), recurrent neural network, or other machine learning model that processes the global context 400. The global context 400 as used below may be replaced with an output of a hidden layer of the encoder. The encoder may be trained to encode a status of the surgical flow, e.g., the current procedure being performed, a listing of all procedures completed as well as the current procedure, or other descriptor of the status of the surgical flow.

The local context 402 of the subject image 200 may include a second set of consecutive images including the subject image 200, such as the last image 200 of the second set of consecutive images or at some intermediate position within the second set of consecutive images. The second set of consecutive images may be a subset of the first set of consecutive images. For example, there may be a first number of images 200 in the first set of consecutive image and a second number of images 200 in the second set of consecutive images, the first number being greater than the second number, such as at least 10, 20, 50, or 100 times the second number. Stated differently, the first set of consecutive images may include all images 200 captured in a first time window of from 1 to 4 minutes, such as from 1.5 to 3 minutes, such as about 2 minutes. In contrast, the second set of images may include all images 200 captured in a second time window of from 1 to 4 seconds, such as from 1.5 to 3 seconds, such as about 2 seconds, the second time window being within the first time window.

The local context 402 may be processed by an encoder 406. In some embodiments, the encoder 406 processes each image 200 in the local context 402 individually. The output of the encoder 406 may be a vector of values characterizing each image 200. For example, the encoder 406 may be the encoder of an encoder-decoder that is trained to receive an image, process the image using the encoder to generate a vector, process the vector to obtain an image that is an attempt to recreate the image.

The outputs of the encoder 406 for the local context 402 may be input to a spatial embedding generator. The spatial embedding generator 408 is trained to receive the vectors from the encoder 406 and output a spatial embedding for each vector that encodes a portion of the information from the vector that is relevant to subsequent stages, e.g., determination of the procedure being performed within the images 200 of the local context 402 were captured.

The spatial embeddings and the label token 404 may be input to a temporal embedding generator 410. The temporal embedding generator 410 processes the spatial embeddings and the label token 404 to generate a temporal embedding that encodes data describing movement represented in the spatial embeddings and therefore in the local context 402.

The temporal embedding and the global context 400 may be input to a decoder 412. The output of the decoder 412 may be a vector representing information (spatial and temporal) included in the images 200 of the global context and the temporal embedding. The vector output from the decoder 412 may be input to a label prediction model 414 that outputs an intermediate label 416 that is input to the fusion model 312 as described above. The intermediate label may have the form described above for the final label 302 or may have a different form. For example, the intermediate layer may include identifier of a procedure and may or may not include a treatment identifier and/or one or more additional levels of sub-procedures.

The encoder 406, the spatial embedding generator 408, and the temporal embedding generator 410 may be characterized as stages in a path from the input to the encoder 406 to the output of the temporal embedding generator 410. The number of values output by each stage may be less than the number of values output by a preceding stage. Likewise, the dimensions of some stages may be less than the dimensions of a preceding stage. For example, the encoder 406 may output a N×M1 array of values, where N is the number of images 200 in the local context 402 and M1 is an integer. The spatial embedding generator 408 may output a N×M2 array of values, where M2 is smaller than N1. The temporal embedding generator 410 may output a vector of M3 values, where M3 is less than M2 times N and may also be less than M2.

Some or all of the encoder 406, spatial embedding generator 408, temporal embedding generator 410, decoder 412, and label prediction model 414 may be trained together or separately. For example, the spatial embedding generator 408 and temporal embedding generator 410 are trained with the decoder 412 such that the embeddings output thereby facilitate generation of a correct label by the decoder 412 and the label prediction model 414.

The encoder 406, spatial embedding generator 408, temporal embedding generator 410, decoder 412, and label prediction model 414 may each be a neural network, deep neural network (e.g., MAMBA network), convolution neural network (including a three-dimensional convolution neural network), recurrent neural network, transformer, multiple linear regression model, random sample consensus regression model, multiple polynomial regression model, support vector regression model, Bayesian neural network, genetic algorithm, long short term memory (LSTM) model, or other type of machine learning model.

Training data entries for training the TVS model 306 may each include a global context 400, a local context 402, and a label token 404 for a video file recording performance of an ophthalmic treatment and a human generated intermediate label 416 corresponding to the action represented in the subject image 200 included in the global context 400 and the local context 402. A training data entry may be processed using the TVS model 306 to obtain an intermediate label 416. The intermediate label 416 output by the TVS model 306 may be compared to the intermediate label 416 of the training data entry and parameters of the TVS model 306 (e.g., of the encoder 406, spatial embedding generator 408, temporal embedding generator 410, decoder 412, and label prediction model 414) may be updated according to the comparison.

The illustrated TVS model 306 has the advantage of learning visual representation in a sequence-modeled manner within a global context, which may help avoid introducing image-specific inductive biases. Furthermore, the local-to-global process assigns specific responsibilities to each model layer, so that the model layers can cooperate better to achieve faster convergence speed and higher performance. Such a hierarchical representation pattern also reduces the total space and time complexity to make the TVS model 306 scalable.

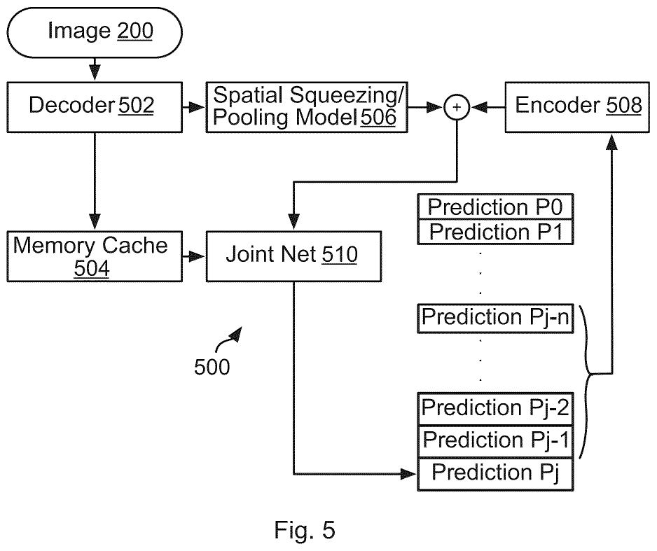

Referring to FIG. 5, the illustrated machine learning model 500 may be used to process images 200 in streaming video. In particular, the machine learning model 500 is well suited where a workflow context 308 is not available such that processing of streaming video is performed in real time without a priori knowledge of an ophthalmic treatment being performed.

The machine learning model 500 may include a decoder 502 that receives an image 200 as an input. The image 200 may be a frame j of a plurality of frames 0 to j of the streaming video. The decoder 502 process the image 200 and generates an output Dj that is stored in a memory cache 504. The memory cache 504 may therefore store outputs Dj−n to Dj for the frames j−n to j, where n is an integer greater than or equal to 1, such as a value from 1 to 100. The output Dj may be a vector or array of values encoding information represented in the image 200. The decoder 502 may be implemented as a decoder according to any approach known in the art.

The output Dj may be input to a spatial squeezing/pooling model 506. The spatial squeezing/pooling model 506 may product an output Sj based on Dj where the number of values of Sj is less than the number of values in Dj. The function of the spatial squeezing/pooling model 506 is to reduce the amount of information in the output Pj relative to the output Dj with the output Sj including information that is more relevant to subsequent steps of the machine learning model 500, e.g., relevant to assigning a label to the image 200 as compared to the input to the spatial squeezing/pooling model 506.

The output Sj may be combined with an output Ej of an encoder 508, e.g., by concatenating, and the combination may be input to a joint net 510. The joint net 510 may be a multimodal deep neural network as known in the art. The encoder 508 may be an encoder according to any approach known in the art, such as the encoder or an encoder-decoder as described above or according to any approach known in the art.

The joint net 510 may further take, as an input, entries from the memory cache 504, such as outputs Dj−n to Dj for frames j−n to j−1 and the image 200. The joint net 510 process the above-mentioned inputs and outputs a prediction Pj, e.g., a predicted label for the procedure being performed when the image 200 was captured. The predicted label may have any of the possible forms described above with respect to the final label 302.

The encoder 508 may take as inputs a set of predictions Pj−n to Pj−1 for frames j−n to j−1 preceding the image 200. The value of n may be between 1 and 100 or some other value. For iterations performed by the machine learning model 500 on frames 0 to n, the output of the encoder 508 may be ignored by the joint net 510. Likewise, for iterations performed by the machine learning model 500 on frames 0 to n, only the outputs Dj−n to Dj−1 that are present in the memory cache 504 will be used.

The decoder 502, spatial squeezing/pooling model 506, and encoder 508 may be a neural network, deep neural network (e.g., MAMBA network), convolution neural network (including a three-dimensional convolution neural network), recurrent neural network, transformer, multiple linear regression model, random sample consensus regression model, multiple polynomial regression model, support vector regression model, Bayesian neural network, genetic algorithm, long short term memory (LSTM) model, or other type of machine learning model.

Training data entries used for training the machine learning model 500 may include an image 200, a set of predictions Pj−n to Pj corresponding to frames preceding the image 200 and the image 200. The predictions Pj−n to Pj may be human generated labels of a procedure being performed during capture of the image 200 and n−1 frames preceding the image 200 in a video stream. The image 200 and predictions Pj−n to Pj−1 of a training data entry may be processed using the machine learning model 500 to obtain a prediction Pj. The prediction Pj output by the machine learning model 500 may be compared to the prediction Pj of the training data entry and parameters of the machine learning model 500 (e.g., of the decoder 502, spatial squeezing/pooling model 506, encoder 508, and joint net 510) may be updated according to the comparison.

The machine learning models 300, 500 and TVS model 306 illustrated are exemplary only. For example, a single machine learning model may be used to process the illustrated inputs to the machine learning models 300, 500 or TVS model 306 rather than the sets of multiple machine learning models illustrated.

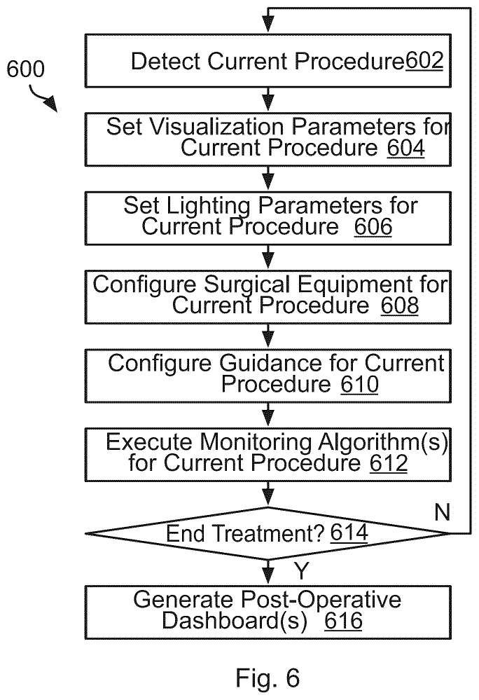

FIGS. 6 to 12 illustrate various use cases for labels generated using either of the machine learning models 300, 500 or other type of machine learning model.

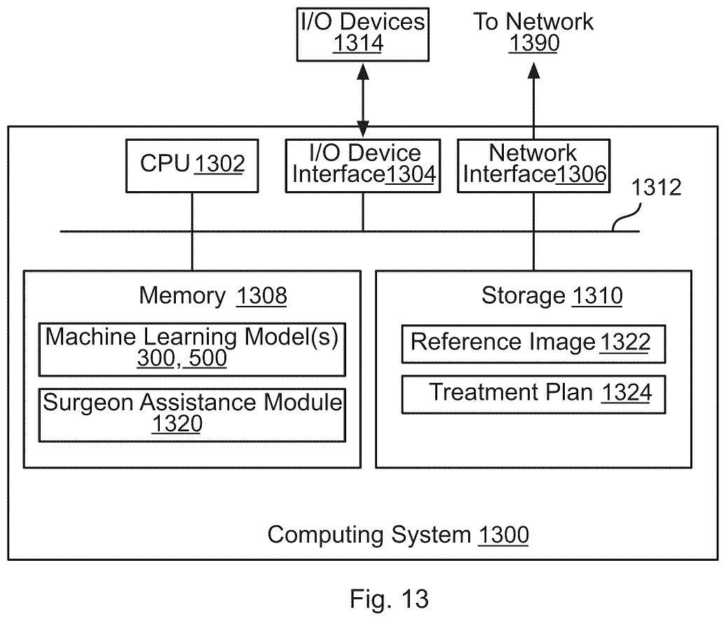

Referring specifically to FIG. 6, the illustrated method 600 illustrates example actions that may be performed using labeled images 200 during an ophthalmic treatment such as cataract surgery, glaucoma surgery (e.g., minimally invasive glaucoma surgery (MIGS), vitrectomy, retinal attachment surgery, retinal membrane peeling, or any other ophthalmic treatment. The method 600 may be performed using the computing system 1300 of FIG. 13.

The method 600 includes detecting, at step 602, the current procedure being performed using one of the machine learning models 300, 500 or other type of machine learning model. The method 600 may include setting, at step 604, one or more visualization parameters corresponding to the current procedure. Visualization parameters may include some or all of filter parameters (temporal and/or spatial), color adjustment, magnification of a particular region of interest (ROI), depth of focus, or other adjustment to images as received from the ophthalmic microscope 102 or operation of the optics of the ophthalmic microscope 102 itself.

The method 600 may include setting, at step 606, one or more lighting parameters corresponding to the current procedure. For example, the ophthalmic microscope 102 may have light sources such as left and right coaxial light sources that are substantially (e.g., within 2 degrees of) an optical axis of the eye 106 and an oblique light source that defines an angle of between 5 and 12, such as between 7 and 10 degrees relative to the optical axis of the eye 106. The intensity, color, polarization, or any other parameters of any these light sources may be set according to the current procedure. Lighting parameters may further include a direction, focus, or other adjustable property for any of the above-referenced light sources. Lighting parameters may refer to modulation (e.g., sinusoidal variation) of any of the color, intensity, polarization, or other property of any of the above-referenced light sources.

The method 600 may include configuring, at step 608, one or more items of surgical equipment according to the current procedure. For example, step 608 may include configuring which item of surgical equipment is controlled by the foot pedals 122, configuring suction pressure of a vacuum pump, configuring an oscillation frequency of cutter in a phaco-vit tool, setting a flow rate for saline, intensity of an inserted illumination tool, or the like.

The method 600 may include configuring, at step 610, guidance according to the current procedure. For example, an overlay may be superimposed over images from the ophthalmic microscope 102 displayed on a display device 118, 120 or a display internal to the ophthalmic microscope 102. The information displayed on the overlay may bet set according to the current procedure. The information displayed may include information from a treatment plan corresponding to the current procedure; markings relative to anatomy of the eye indicating an incision location or providing an alignment guide; information obtained from processing images from the ophthalmic microscope (e.g., measurements of anatomy); information describing the state of operation of surgical equipment; or other information. Step 610 may include outputting a message to one or more members of a surgical team on one or more other devices, e.g., instructions regarding preparation for a next procedure in the ophthalmic treatment.

The method 600 may include executing, at step 612, one or more monitoring algorithms corresponding to the current procedure. A monitoring algorithm may output alerts or execute remediating actions in response to an unsafe condition. A monitoring algorithm may monitor the state of surgical equipment and alert the surgeon if the state is outside of acceptable boundaries. The monitoring algorithm may monitor movements of instruments and alert the surgeon if the instruments are near (e.g., within 0.1 millimeters) or outside of an acceptable operating envelope. The monitoring algorithm may monitor anatomy represented in images 200 and alert the surgeon of the anatomy indicates that an unsafe condition is present.

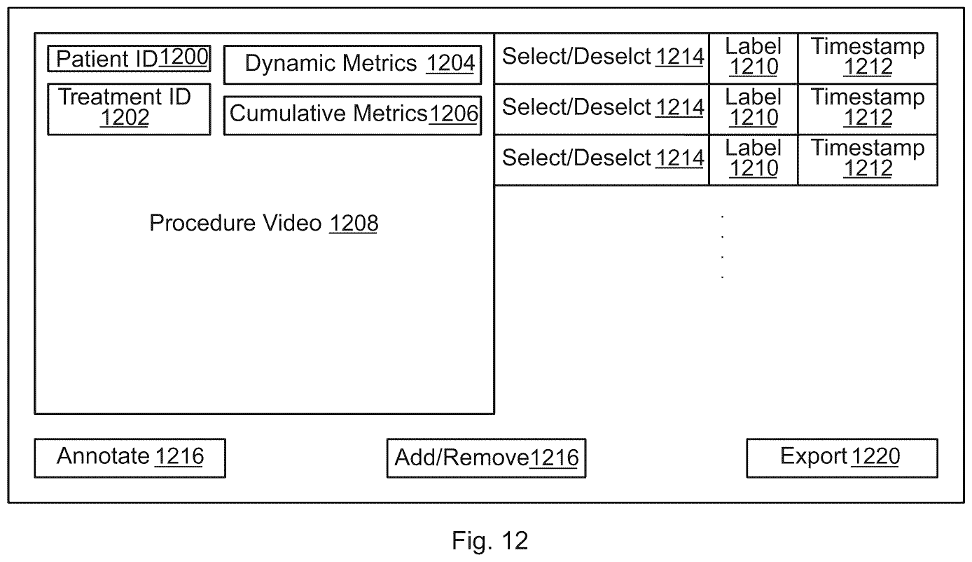

Some or all of steps 604 to 614 may be performed for each procedure. In particular, not all procedures will require performance of all of steps 604 to 614. Once all of the procedures of an ophthalmic treatment are found at step 614 to have been completed, the method 600 may include generating, at step 616, and displaying a post-operative dashboard. The dashboard may enable access to segments of video captured during the ophthalmic treatment and segmented using one of the machine learning models 300, 500 or another machine learning model. An example dashboard is discussed below with reference to FIGS. 11 and 12.

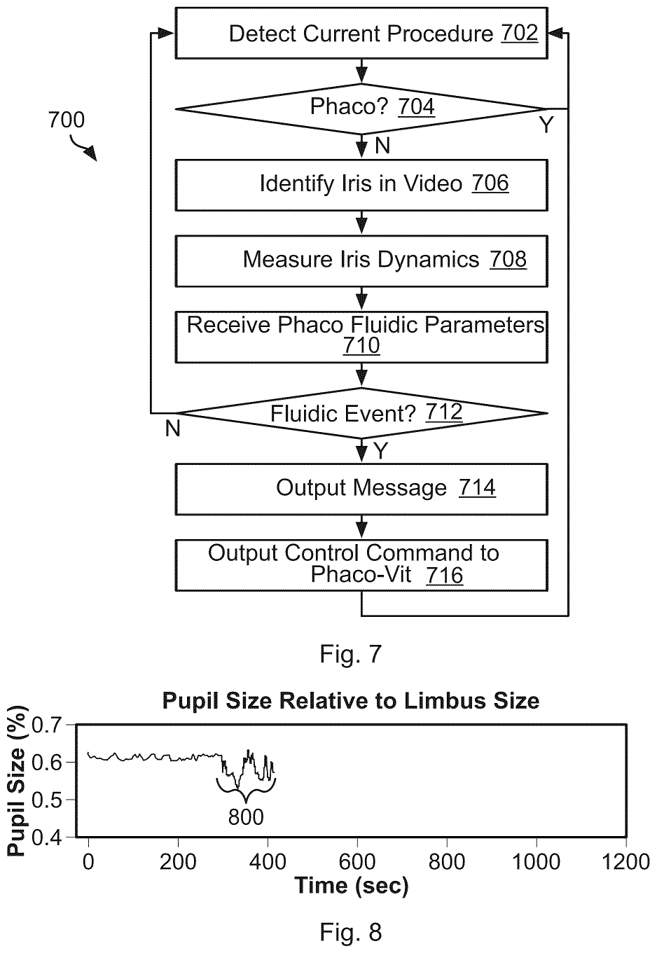

FIG. 7 illustrates an example method 700 that is a specific application of the method 600 to a cataract surgery. The method 700 may be performed using the computing system 1300 of FIG. 13.

The method 700 may include detecting, at step 702, the current procedure, such as using one of the machine learning models 300, 500 or other machine learning model. If the current procedure is found, at step 704, to be a phaco-emulsification step (removal of the crystalline lens), some or all of the subsequent steps of the method 700 may be performed. If not, then the method 700 may continue at step 702. If a procedure other than phaco-emulsification is detected at step 702, another method corresponding to that procedure may instead be performed.

For example, the method 700 may include identifying, at step 706, representations of the iris of the eye 106 in video captured by the ophthalmic microscope 102, which may be the same video used to detect the current procedure at step 702.

The method 700 may include measuring, at step 708, iris dynamics. For example, pupil size of the iris may be measured for a plurality of images 200 in the video along with limbus size (see plot of pupil size relative to limbus size over time in FIG. 8). Variation in in pupil size relative to the limbus diameter of the eye over time may be evaluated at step 708, such as the rate of change of pupil size relative to the limbus diameter.

The method 700 may further include receiving, at step 710, fluidic parameters for a phaco-vit tool, such as measurements of intra-ocular pressure (IOP), vacuum pressure, aspiration (e.g., aspiration flow rate), or other values.

The method 700 may include evaluating, at step 712, the iris dynamics and the fluidic parameters to determine whether a fluidic event is indicated. A fluidic event may, for example, include occlusion of a tip of a phaco-vit tool. For example, referring to FIG. 8, the rate of change in region 800 being above a threshold along with fluidic parameters exceeding one or more thresholds (e.g., IOP rising or being above an IOP threshold, vacuum pressure rising or being above a vacuum pressure threshold) may indicate a fluidic event. If a fluidic event is not detected, processing may continue at step 702, e.g., to evaluate whether the phaco-emulsification step is still being performed.

If a fluid event is detected, one or more actions may be performed such as outputting, at step 714, a message to the surgeon 104 indicating that a fluidic event is occurring. If a fluid event is detected, the method 700 may include outputting, at step 716, one or more control commands to a phaco vit machine. For example, the amount of vacuum pressure may be briefly (e.g., less than 100 milliseconds) increased to clear the occlusion or turned off to enable the surgeon 104 to clear the occlusion.

Following one or both of step 714 and 716, processing may continue at step 702, e.g., to evaluate whether the phaco-emulsification step is still being performed.

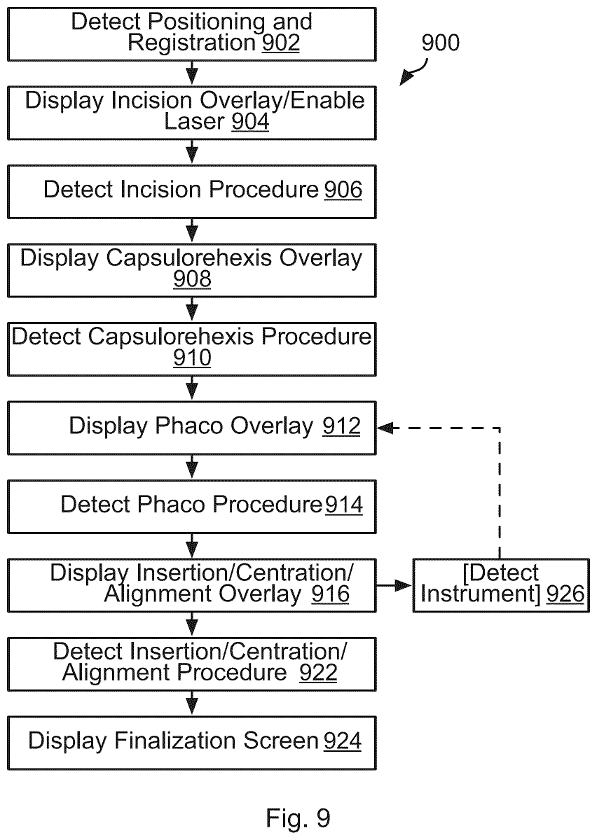

FIG. 9 illustrates an example method 900 that may also be an application of the method 600 to a cataract surgery. The method 900 may be performed using the computing system 1300 of FIG. 13. In the description of the method 900 detecting of a particular procedure may be understood as being performed using machine learning model 300, 500 or other machine learning model. In the description of the method 900, detecting completion of a procedure may be detected explicitly or may be implicitly detected in response to detecting performance of a different procedure, e.g., a next procedure in a treatment plan for the cataract surgery.

The method 900 may include detecting, at step 902, performance of a positioning procedure. The positioning procedure may include positioning the ophthalmic microscope 102 in a desired relative position to the eye 106 of the patient receiving cataract surgery, such as having the optical axis of the eye 106 aligned within a tolerance of an optical axis of the ophthalmic microscope 102. Step 902 may further include detecting completion of a registration step. The registration step may include evaluating video images received from the ophthalmic microscope 102 relative to a reference image. Registration may include identifying the anatomy represented in the video images and matching the anatomy to anatomy represented in the reference image. Relative positions of the anatomy in the video images and reference images may be used to determine a transform of coordinates in the reference image and coordinates in the video image. In this manner, Overlays defined relative to the reference image may be applied to the video images as discussed in greater detail below.

In response to detecting positioning and registration, the method 900 may include displaying, at step 904, an incision overlay. Step 904 may further include activating a laser where a laser is used to make the incision.

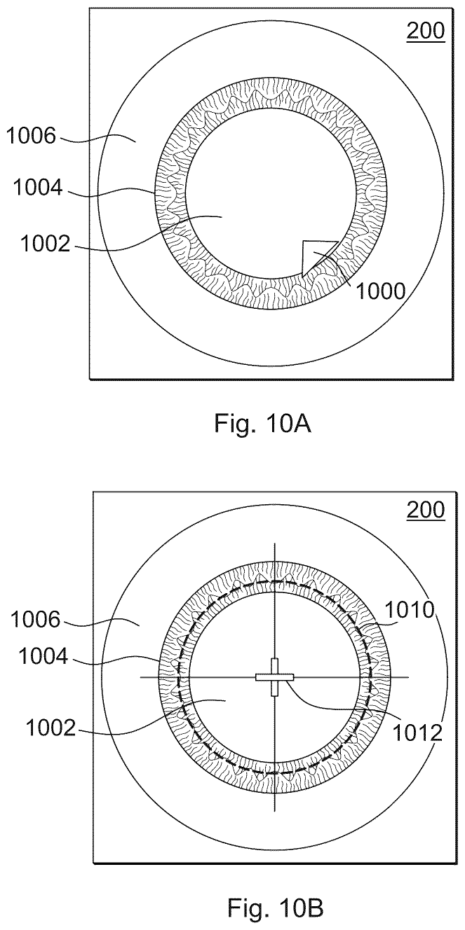

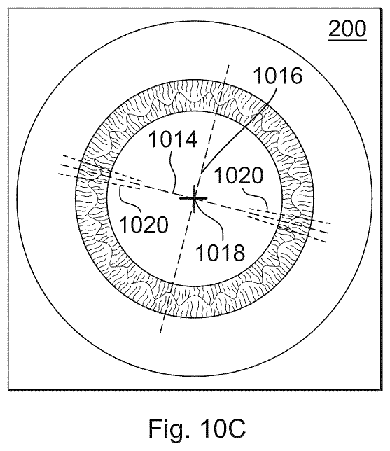

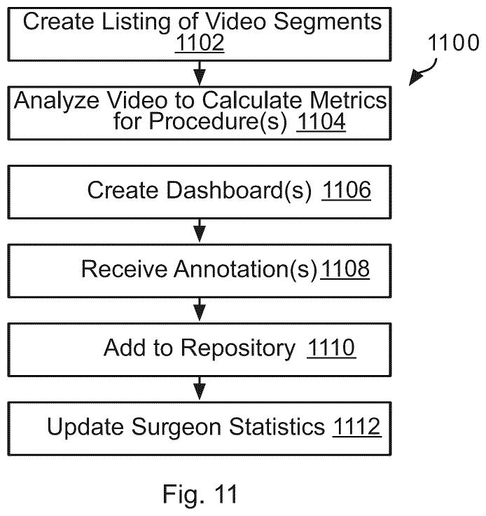

For example, referring to FIG. 10A, an incision guide 1000 may be displayed on an image 200 depicting the eye 106. The image 200 may include representations of the cornea 1002, limbus 1004, and sclera 1006 of the eye 106. The incision guide 1000 may be placed at a location in the 1002 near the limbus 1004. There may be multiple incision guides 1000, such as a primary incision guide 1000 and one or more secondary incision guides 1000.

Referring again to FIG. 9, the method 900 may include detecting, at step 906 completion of the incision procedure and, in response, displaying, at step 908, a capsulorehexis overlay to guide the surgeon 104 in performing capsulorhexis, e.g., cutting an opening in the capsular bag of the eye 106 to facilitate removal of the crystalline lens.

For example, referring to FIG. 10B, while still referring to FIG. 9, the capsulorhexis overlay may include a rhexis element 1010 defining a perimeter of the rhexis. The rhexis element 1010 may be a circle centered on a center 1012, which may be approximately (e.g., within 0.1 millimeter) intersected by an optical axis of the eye 106.

Referring again to FIG. 9, the method 900 may include detecting, at step 910, completion of the capsulorhexis procedure, and in response, displaying, at step 912, a phacoemulsification overlay. A phacoemulsification overlay may display information such as the vacuum pressure of a phaco-vit too. IOP, elapsed duration of the phacoemulsification process, or other data that may facilitate performance of the phacoemulsification procedure.

The method 900 may include detecting, at step 914, completion of the phacoemulsification procedure and/in response, displaying, at step 916, an overlay for facilitating some or all of insertion of a lens (e.g., an intraocular lens (IOL)), centration of the lens, and alignment of a toric axis of the lens.

For example, referring to FIG. 10C, the overlay of step 914 may include some or all of a label 1014 indicating the orientation of a toric axis of the lens, a label 1016 that along with the label 1014 indicates a center of the lens (e.g., a line perpendicular to the toric axis), a label 1018 indicating the optical axis of the eye 106, and labels 1020 indicating an acceptable range of angles for the toric axis of the lens relative to the axis of astigmatism of the eye 106. The optical axis of the eye 106, the position of the center of the lens, and the orientation of the axis may be determined by evaluating the images 200 using any approach known in the art.

Referring again to FIG. 9, the method 900 may include detecting, at step 922, completion of insertion, centration, and alignment of the toric axis of the lens and/in response, displaying, at step 924, a finalization screen. The finalization screen may enumerate final steps to complete the cataract surgery, present metrics characterizing the cataract surgery (e.g., elapsed time for one or more procedures of the cataract surgery), or other information.

Note that in some embodiments, the method 900 may include detecting a return to a procedure that was previously detected as completed. For example, at some point following step 914, the method 900 may include detecting, at step 926, insertion of a phaco-vit instrument. For example, a machine learning model 300, 500 or other machine learning model may detect one or more images 200 as corresponding to the phaco-emulsification procedure. In response, the method 900 may return to step 912 with display of the phaco-emulsification overlay. Instruments corresponding to other procedures may likewise be identified and invoke return to displaying the overlay for the other procedures in the like manner.

FIG. 11 illustrates a method 1100 for generating a post-operative dashboard, such as the dashboard shown in FIG. 12. The dashboard can serve as a central data hub that binds multimodality data stream (such as those from a device such as the ophthalmic microscope 102) with the time-stamped surgical video segments and analysis. The method 1100 may be performed as part of step 616 of the method 600 with respect to video captured during an ophthalmic treatment (“the video file”) and including the images 200 that have been labeled using one of the machine learning models 300, 500 or other machine learning model.

The method 1100 may include creating, at step 1102, a listing of video segments. The method 1100 may be performed using the computing system 1300. For example, for each procedure of an ophthalmic treatment, a consecutive set of images 200 from the video that are labeled as corresponding to that procedure may be used to create a video segment for that procedure. The segment may include a separate video file, a reference to a start and end time within the video file, or indexes of first and last images of the consecutive set of images in the video file.

The method 1100 may include analyzing, at step 1104, the video segments to calculate metrics for the procedures corresponding to the video segments. Step 1104 may further include analyzing other available data for the ophthalmic treatment, such as parameters controlling operation of surgical equipment during the ophthalmic treatment, surgeon inputs to control surgical equipment during the ophthalmic treatment, or other available data collected during the ophthalmic treatment. For example, the metrics may include dynamic parameters such as some or all of the following non-limiting examples:
Patient biometry, such as pupil diameter and iris dynamics
Precision of toric IOL alignment in the eye.
Angle of the cornea incision site on the eye
Shape and centration of the capsulorhexis
Trajectory and motion of an instrument (e.g., Phaco probe)

Step 1104 may include calculating one or more cumulative metrics for a procedure or an entire ophthalmic treatment. For example, the cumulative metrics may include some or all of the following non-limiting examples:
Total case time
Active surge mitigation actuations (ASM)
Total aspiration time
Estimated fluid aspirated
Average longitudinal power (e.g., FP3)
Total longitudinal power-on time
Average torsional amplitude (e.g., FP3)
Total torsional amplitude-on time
Equivalent average torsional amplitude (e.g., FP3)
Equivalent average ultrasonic power (e.g., FP3)
Cumulative dissipated energy (CDE)
Ultrasonic total time
Instrument to limbus distance
Instrument to pupil distance
Amount of instrument movement within a time duration
Time-to-motion statistics
Energy use efficiency

The method 1100 may include creating 1106 a dashboard including the listing of video segments and representations of one or more of the metrics calculated at step 1104.

For example, referring to FIG. 12, a dashboard may display such information as a patient identifier 1200 and an identifier 1202 of the ophthalmic treatment. The dashboard may include representations of one or more dynamic metrics 1204 and/or one or more cumulative metrics 1206. The dashboard may include a window displaying video 1208 for a procedure, e.g., a video segment from step 1102. The representations of the one or more dynamic metrics 1204 and/or one or more cumulative metrics 1206 may be synchronized with the video 1208, e.g., the dynamic metrics 1204 corresponding to a currently displayed frame of the video 1208 and the cumulative metrics 1206 corresponding to the values thereof at a time corresponding to the currently displayed frame of the video 1208. The metrics 1204, 1206 displayed may correspond to the procedure with which the video segment being displayed is labeled.

The dashboard may include a representation of the listing of video segments from step 1102. For example, the dashboard may include, for each video segment, a label 1210 and a timestamp 1212. The label 1210 may be a label assigned to the video segment by a machine learning model 300, 500 or other machine learning model an the timestamp 1212 may be a time within the video file corresponding to a first frame of the video segment. Each entry in the listing may include one or more interface elements 1214 for managing the video segment, such as an interface element for selecting or deselecting the video segment as the object of an operation invoked by another interface element, for playback, or other purpose.

The dashboard may include interface elements for invoking one or more actions with respect to a video segment, the video file, or a data object including the video file along with other information, such as the metrics from step 1104. For example, interface element 1216 may invoke an interface for receiving an annotation and invoking addition of the annotation to a video segment. The annotation may be received as typed text, speech that is transcribed to text, graphical additions to one or more frames of the video segment, or other type of annotation.

The dashboard may include an interface element 1216 that, when selected invokes receiving an instruction to add or remove a video segment and then processes the video file to add or remove the video segment as instructed. For example, a user may join two video segments to make a single video segments, adjust the starting frame of a video segment to make a video segment shorter or longer, or divide a video segment into two video segments.

The dashboard may include an interface element 1220 that, when selected, invokes exporting of the video segments, any annotations, and the metrics, such as to a database, to a messaging modality (text, email), or other destination. The interface element 1220 may include elements that enables user to quickly review case videos and to conduct case search.

The interface elements on the dashboard may be changed in response to user selection of an entry in the listing of video segments. In particular, the dynamic and/or cumulative metrics 1204, 1206 may be changed to correspond to those calculated for the video segment of the selected entry.

Referring again to FIG. 11, the method 1100 may include receiving, at step 1108, one or more annotations to the dashboard. The method 1100 may include adding, at step 1110, a treatment record to a database, the treatment record may include the video file, any annotations, and the metrics to a repository. The repository may be a database storing treatment records for a single surgeon 104 or a plurality of surgeons 104.

The method 1100 may include updating, at step 1112, statistics for the surgeon 104 according to the treatment record, such as one or more metrics from the treatment record. For example, any of the cumulative metrics for a plurality of ophthalmic treatments performed by the surgeon 104 may be averaged or statistically characterized (e.g., maximum, minimum, standard deviation, etc.). The statistics for multiple surgeons may be compared to one another to obtain a ranking of surgeons. Rankings may be used as part of incentive programs or gamification program to improve patient outcomes. For example, leader board may be updated according to the statistics.

Labeled video segments for procedures along with any of the metrics described herein could be stored in a central data hub. Labeled video segments may be added to a pool for a group or communities formed by surgeons. The pool can enable better knowledge sharing between surgeons such as showcasing different surgical techniques, or enable competition, such as leaderboard of surgical metrics, such as time-to-motion statistics, energy use efficiency, percent of complicated cases, etc.

Labeled video segments for an ophthalmic treatment may alternatively or additionally be used for various other purposes. For example, step 1104 may include calculating metrics that may then be aggregated in order to facilitate correlation between the metrics and patient outcomes. For example, metrics of a rhexis procedure may include such metrics as size, location, and roundness. The metrics of the rhexis procedure may be analyzed from a labeled video segment corresponding to after implantation of an IOL in the patient eye. The labeled video segment corresponding to after implantation of an IOL may be analyzed to determine metrics of IOL positioning, such as centration, and toric axis alignment. Through a patient data portfolio management system, the post-op refractive outcome of the patient data can be linked to any of the above-described metrics. With enough data aggregation, a surgeon may conduct a research study to understand how metrics of rhexis and IOL position can impact patient visual acuity outcomes.

In another example, a patient outcome may be retrospectively tagged as, for example, ‘optimal outcome’, or ‘suboptimal outcome.’ The labeled video segments of certain procedures can be group according to those tags. Grouped video clips can improve the learning experience of junior surgeons or fellows, to facilitate understanding of what surgical techniques can lead to ‘optimal outcome’ for the patient.

In another example, the labeled video segments for an ophthalmic treatment may be labeled with results of intra-operative analysis. For example, some or all of the following conditions may be profiled and referenced by tags:
Myopic eye having long axial length showing reverse pupillary block
A floppy iris
A small pupil
Use of a mechanical pupil dilation device

These tags and/or tags corresponding to other pupil conditions or other ocular conditions may be associated with the labeled video segments of a procedure. A surgeon is thereby enabled to quickly retrieve relevant cases relating to any of the conditions referenced by the tags, such as for the purpose of teaching.

FIG. 13 illustrates an example computing system 1300. The ophthalmic microscope 102 and the display devices 118, 120 may incorporate a computing device having some or all of the attributes of the computing system 1300.

As shown, computing system 1300 includes a central processing unit (CPU) 1302, one or more I/O device interfaces 1304, which may allow for the connection of various I/O devices 1314 (e.g., keyboards, displays, mouse devices, pen input, etc.) to computing system 1300, network interface 1306 through which computing system 1300 is connected to network 1390, a memory 1308, storage 1310, and an interconnect 1312.

CPU 1302 may retrieve and execute programming instructions stored in the memory 1308. Similarly, CPU 1302 may retrieve and store application data residing in the memory 1308. The interconnect 1312 transmits programming instructions and application data, among CPU 1302, I/O device interface 1304, network interface 1306, memory 1308, and storage 1310. CPU 1302 is included to be representative of a single CPU, multiple CPUs, a single CPU having multiple processing cores, and the like.

Memory 1308 is representative of a volatile memory, such as a random access memory, and/or a nonvolatile memory, such as nonvolatile random access memory, phase change random access memory, or the like. As shown, memory 1308 may store executable code implementing the machine learning models 300, 500 or other machine learning model for labeling video segments as described above. The memory 1308 may store a surgeon assistance module 1320 configured to perform some or all of the methods 600, 700, 900, 1100.

Storage 1310 may be non-volatile memory, such as a disk drive, solid state drive, or a collection of storage devices distributed across multiple storage systems. Storage 1310 may optionally store a reference image 1322 and a treatment plan 1324 for an ophthalmic treatment as defined above.

### Example Embodiments

In certain embodiments, a system comprises an ophthalmic microscope configured to capture video of an ophthalmic treatment and a computer system coupled to the ophthalmic microscope. The computer system is configured to receive a treatment plan for the ophthalmic treatment; process the video and the treatment plan using a machine learning model to divide the video into a plurality of video segments, each video segment of the plurality of video segments corresponding to a procedure of a plurality of procedures included in the ophthalmic treatment; and control surgical equipment according to an output of the machine learning model.

In certain embodiments, a method comprises receiving, by a computer system, a treatment plan for an ophthalmic treatment; receiving, by the computer system, from an ophthalmic microscope, video of an ophthalmic treatment; and processing, by the computer system, the video and the treatment plan using a machine learning model to divide the video into a plurality a plurality of video segments, each video segment of the plurality of video segments corresponding to a procedure of a plurality of procedures included in the ophthalmic treatment.

In certain embodiments, a system comprises an ophthalmic microscope configured to stream video of an ophthalmic treatment, and a computer system coupled to the ophthalmic microscope. The computer system is configured to process the video by, for each frame of at least a portion of frames in the video, process each frame using a first machine learning model to obtain a first machine learning model output for each frame; process, using a second machine learning model a combination of (a) the first machine learning model output for each frame, (b) first machine learning model outputs for a plurality of frames of the video preceding each frame, (c) labels for the plurality of frames of the video preceding each frame previously output by the second machine learning model; and obtain, from the second machine learning model processing (a), (b), and (c), a label for each frame, the label identifying a procedure of a plurality of procedures of the ophthalmic treatment.

### Additional Considerations

The preceding description is provided to enable any person skilled in the art to practice the various embodiments described herein. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The various illustrative logical blocks, modules and circuits described in connection with the present disclosure may be implemented or performed with a general purpose processor, a digital signal processor (DSP), an application specific integrated circuit (ASIC), a field programmable gate array (FPGA) or other programmable logic device (PLD), discrete gate or transistor logic, discrete hardware components, or any combination thereof designed to perform the functions described herein. A general-purpose processor may be a microprocessor, but in the alternative, the processor may be any commercially available processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

A processing system may be implemented with a bus architecture. The bus may include any number of interconnecting buses and bridges depending on the specific application of the processing system and the overall design constraints. The bus may link together various circuits including a processor, machine-readable media, and input/output devices, among others. A user interface (e.g., keypad, display, mouse, joystick, etc.) may also be connected to the bus. The bus may also link various other circuits such as timing sources, peripherals, voltage regulators, power management circuits, and the like, which are well known in the art, and therefore, will not be described any further. The processor may be implemented with one or more general-purpose and/or special-purpose processors. Examples include microprocessors, microcontrollers, DSP processors, and other circuitry that can execute software. Those skilled in the art will recognize how best to implement the described functionality for the processing system depending on the particular application and the overall design constraints imposed on the overall system.

If implemented in software, the functions may be stored or transmitted over as one or more instructions or code on a computer-readable medium. Software shall be construed broadly to mean instructions, data, or any combination thereof, whether referred to as software, firmware, middleware, microcode, hardware description language, or otherwise. Computer-readable media include both computer storage media and communication media, such as any medium that facilitates transfer of a computer program from one place to another. The processor may be responsible for managing the bus and general processing, including the execution of software modules stored on the computer-readable storage media. A computer-readable storage medium may be coupled to a processor such that the processor can read information from, and write information to, the storage medium. In the alternative, the storage medium may be integral to the processor. By way of example, the computer-readable media may include a transmission line, a carrier wave modulated by data, and/or a computer readable storage medium with instructions stored thereon separate from the wireless node, all of which may be accessed by the processor through the bus interface. Alternatively, or in addition, the computer-readable media, or any portion thereof, may be integrated into the processor, such as the case may be with cache and/or general register files. Examples of machine-readable storage media may include, by way of example, RAM (Random Access Memory), flash memory, ROM (Read Only Memory), PROM (Programmable Read-Only Memory), EPROM (Erasable Programmable Read-Only Memory), EEPROM (Electrically Erasable Programmable Read-Only Memory), registers, magnetic disks, optical disks, hard drives, or any other suitable storage medium, or any combination thereof. The machine-readable media may be embodied in a computer-program product.

A software module may comprise a single instruction, or many instructions, and may be distributed over several different code segments, among different programs, and across multiple storage media. The computer-readable media may comprise a number of software modules. The software modules include instructions that, when executed by an apparatus such as a processor, cause the processing system to perform various functions. The software modules may include a transmission module and a receiving module. Each software module may reside in a single storage device or be distributed across multiple storage devices. By way of example, a software module may be loaded into RAM from a hard drive when a triggering event occurs. During execution of the software module, the processor may load some of the instructions into cache to increase access speed. One or more cache lines may then be loaded into a general register file for execution by the processor. When referring to the functionality of a software module, it will be understood that such functionality is implemented by the processor when executing instructions from that software module.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A system comprising:
an ophthalmic microscope configured to capture video of an ophthalmic treatment; and
a computer system coupled to the ophthalmic microscope, the computer system configured to:
receive a treatment plan for the ophthalmic treatment;
process the video and the treatment plan using a machine learning model to divide the video into a plurality of video segments, each video segment of the plurality of video segments corresponding to a procedure of a plurality of procedures included in the ophthalmic treatment; and
control surgical equipment according to an output of the machine learning model.

2. The system of claim 1, wherein the machine learning model comprises:
an embedding generator configured to generate an embedding based on the treatment plan;
a temporal video segmentation model configured to generate an intermediate label for each frame of the video; and
a fusion model configured to combine the embedding and the intermediate label to generate a final label for each frame of the video, the intermediate label and final label each identifying a procedure of plurality of procedures.

3. The system of claim 2, wherein the temporal video segmentation model is configured to, for each frame of the video, process a local context and a global context for each frame of the video.

4. The system of claim 3, wherein the local context for each frame of the video includes a first set of consecutive frames of the video including each frame of the video and the global context includes a second set of consecutive frames of the video including each frame of the video, the second set of consecutive frames being larger than the first set of consecutive frames.

5. The system of claim 4, wherein the second set of consecutive frames is at least ten times larger than the first set of consecutive frames.

6. The system of claim 4, wherein the temporal video segmentation model comprises:
a one or more first machine learning models configured to process the local context and produce an embedding;
a second machine learning model configured to process the embedding and the global context; and
a third machine learning model configured to process an output of the second machine learning model to obtain the intermediate label for each frame of the video.

7. The system of claim 1, wherein the ophthalmic treatment is a cataract surgery.

8. The system of claim 7, wherein the plurality of procedures include incision, rhexis, phaco-emulsification, insertion, centration, and alignment.

9. The system of claim 1, wherein the computer system is configured to select information to display during the ophthalmic treatment according to an output of the machine learning model.

10. The system of claim 1, wherein the computer system is configured to control surgical equipment according to the output of the machine learning model by controlling operation of a phaco-vit tool.

11. A method comprising:
receiving, by a computer system, a treatment plan for an ophthalmic treatment;
receiving, by the computer system, from an ophthalmic microscope, video of an ophthalmic treatment; and
processing, by the computer system, the video and the treatment plan using a machine learning model to divide the video into a plurality a plurality of video segments, each video segment of the plurality of video segments corresponding to a procedure of a plurality of procedures included in the ophthalmic treatment.

12. The method of claim 11, wherein processing, by the computer system, the video and the treatment plan using a machine learning model comprises:
processing, by the computer system, the treatment plan using an embedding generator to generate an embedding;
processing, by the computer system, the video using a temporal video segmentation model to generate an intermediate label for each frame of the video; and
processing, by the computer system, the embedding and the intermediate label using a fusion model configured to combine the embedding and the intermediate label to generate a final label for each frame of the video, the intermediate label and final label each identifying a procedure of plurality of procedures.

13. The method of claim 12, wherein processing the video using the temporal video segmentation model comprises, for each frame of the video, processing, by the computer system a local context and a global context for each frame of the video, wherein the local context of each frame of the video includes a first set of consecutive frames of the video including each frame of the video and the global context includes a second set of consecutive frames of the video including each frame of the video, the second set of consecutive frames being larger than the first set of consecutive frames.

14. The method of claim 13, wherein the second set of consecutive frames is at least ten times larger than the first set of consecutive frames.

15. The method of claim 11, wherein the ophthalmic treatment is a cataract surgery.
